(19) United States
(12) Patent Application Publication
(10) Pub. No.: US 2006/0184170 A1
(43) Pub. Date: Aug. 17, 2006

(54) Bone fixation apparatus

(75) Inventors: John Kapitan, Waxhaw, NC (US); J. Walt Simmons, San Antonio, TX (US); David McCord, Nashville, TN (US)

Correspondence Address: CALFEE HALTER & GRISWOLD, LLP, 800 SUPERIOR AVENUE, SUITE 1400, CLEVELAND, OH, 44114, US

(73) Assignee: Altiva Corporation, Charlotte, NC (US)

(21) Appl. No.: 11/173,495

(22) Filed: Jul. 1, 2005

Related U.S. Application Data
(60) Provisional application No. 60/652,570, filed on Feb. 14, 2005.

(51) Int. Cl. A61F 2/30

(52) U.S. Cl. 606/61

(57) ABSTRACT

A polyaxial bone fixation system minimizes the number of parts used to engage a bone bolt in various angular orientations relative to an elongated plate or rods, and also permits for nearly infinite positioning of two or more bone bolts relative to each other along the length of an elongated plate or rods.

### PRIORITY CLAIM

This application claims priority to U.S. Provisional Patent Application 60/652,570, filed Feb. 14, 2005, which is incorporated herein by reference, in its entirety.

### FIELD OF THE INVENTION

The present invention relates to surgical apparatuses, and more particularly to improved bone fixation apparatuses with improved positioning and locking capabilities. The apparatuses are particularly useful for spinal fixation.

### BACKGROUND OF THE INVENTION

A variety of clinical conditions require the use of fixation devices, such as apparatuses that include screws and connecting rods or plates, to achieve fixation of bones relative to each other. Numerous spinal fixation procedures involve the temporary and sometimes permanent use of such devices. Procedures for attaching fixation devices to the spine involve accessing the spine either from the back of the patient (posterior) or through the abdominal cavity (anterior). In a typical fixation procedure, bone bolts are used to affix and secure one or more rods or plates to adjacent vertebra within one or more of the cervical, thoracic, or lumbar regions of the spine, so as to achieve fixation of one or more vertebra relative to others. Fixation devices comprising bolts and plates or rods are well known in the art.

It has been found desirable for bone fixation systems to accommodate angular orientation of a bolt or other anchor in multiple axes (“polyaxial”) relative to the fixation rod or plate and to other fixation components of the implant system. The capability of polyaxial fixation of one or more bone bolts in a fixation system is desirable such that each bolt can be placed in an optimal position for anchoring, enabling the surgeon to accommodate for uneven bone surfaces and any abnormalities that would influence bolt positioning. Polyaxial positioning is also deemed desirable because it should reduce the likelihood that the bone fixation system will loosen or pull out during use.

Attempts in the art to provide for polyaxial positioning of bone bolts in bone fixation systems has led to devices that include a number of components that make attachment in the surgical field complicated and cumbersome. Such systems often require the surgeon to choose between several different component designs to configure a system appropriate to optimize fixation in a particular patient. The number and range of device components required in many polyaxial systems introduce undesirable time and complexity into the surgery. In addition, such systems also provide for only a finite number of positioning options for each of the bone bolts relative to each other along the length of the fixation rod or plate. This aspect of existing fixation systems imposes further limits on the surgeon's ability to achieve desired fixation.

There is a need remaining in the art for a polyaxial bone fixation system that minimizes the number of parts used to engage a bone bolt in various angular orientations relative to an elongated plate or rods, and that also permits for nearly infinite positioning of two or more bone bolts relative to each other.

### SUMMARY OF THE INVENTION

In various embodiments, bone fixation locking systems and devices are provided that permit the relative fixation of two or more bone bolts. The bone fixation locking systems allow each bone bolt to be oriented in bone tissue at a wide range of axial positions relative to the plane of an elongated plate or rods, and the axes of other bone bolts. The bone fixation locking systems also allow each bone bolt to be positioned at an infinite number of positions along the length of an elongated plate or rod. According to the various embodiments, the bone fixation locking systems include a locking sleeve that has opposing upper and lower walls with interior and exterior faces, opposing first and second sidewalls, and opposing first and second opened ends that define an aperture through the locking sleeve. The upper and lower walls of the locking sleeve each have through holes that share a common axis, and the upper wall is defined on the exterior face of the upper wall by a spherical convex outer perimeter.

### BRIEF DESCRIPTION OF THE DRAWINGS

For a further understanding of the nature and objects of the present invention, reference should be made to the following detailed description taken in conjunction with the accompanying drawings, in which like parts are given like reference numerals, and wherein:

FIG. 1 is an exploded perspective view showing a compression washer with a threaded nut portion, a locking sleeve, an elongated plate, and a bone bolt, aligned but not engaged, according to one illustrated embodiment of the apparatus of the present invention;

FIG. 2 is a perspective view of one embodiment of a bone bolt according to the present invention;

FIG. 3 is a perspective view of one embodiment of an elongated plate according to the present invention;

FIG. 4 is a perspective view of one embodiment of an elongated plate having a through-hole at one end according to the present invention;

FIG. 5 is a perspective view of one embodiment of an elongated plate having a through-hole at one end according to the present invention, wherein the through-hole is defined by a spherical convex outer perimeter on the upper surface;

FIG. 6A is a perspective view of a locking sleeve according to one embodiment of the present invention;

FIG. 6B is a perspective view of the exterior face of the upper wall of a locking sleeve showing a through-hole defined by a spherical convex outer perimeter on the exterior face of the upper wall according to one embodiment of the present invention;

FIG. 6C is a perspective view of the exterior face of the lower wall of a locking sleeve showing a through-hole defined by a spherical concave inner perimeter on the exterior face of the lower wall according to one embodiment of the present invention;

FIG. 6D is a perspective view of the exterior face of the upper wall of a locking sleeve showing a through-hole defined by a elliptical convex outer perimeter on the exterior face of the upper wall according to one embodiment of the present invention;

FIG. 7 is a side view of one opened end side of a locking sleeve according to one embodiment of the present invention showing through-holes defined by a spherical convex outer perimeter on the exterior faces of the upper and lower walls;

FIG. 8A is a perspective view of a compression washer showing a spherical concave lower surface according to one embodiment of the present invention;

FIG. 8B is a perspective view of a compression washer showing a threaded nut portion according to one embodiment of the present invention;

FIG. 8C is a cut-away side view of one embodiment of a compression washer with a threaded nut portion according to one embodiment of the present invention;

FIG. 9A is a perspective view showing engagement between the spherical concave lower surface of a compression washer and the spherical convex outer perimeter on a locking sleeve through-hole according to an embodiment of the present invention;

FIG. 9B is a perspective view showing engagement between the spherical concave lower surface of a compression washer and a spherical convex outer perimeter on an elongated plate through-hole according to an embodiment of the present invention;

FIG. 9C is a side view of a locking sleeve sidewall showing engagement between the spherical concave lower surface of a first compression washer and the spherical convex outer perimeter on an upper through-hole, and engagement between the spherical concave lower surface of a second compression washer and a spherical convex outer perimeter on a lower through-hole according to an embodiment of the present invention;

FIG. 10A is a perspective view showing engagement between a compression washer with a threaded nut portion, a locking sleeve shown in FIG. 6, an elongated plate, and a bone bolt according to an embodiment of the present invention;

FIG. 10B is a perspective view of the bone fixation apparatus shown in FIG. 10A showing compression of the locking sleeve according to an embodiment of the present invention;

FIG. 11A is a perspective view showing engagement between a compression washer with a threaded nut portion, a second compression washer, a locking sleeve as shown in FIG. 7, an elongated plate, and a bone bolt according to an embodiment of the present invention;

FIG. 11B is a perspective view of the bone fixation apparatus shown in FIG. 11A showing compression of the locking sleeve according to an embodiment of the present invention;

FIG. 12 is a cut-away side view showing three of the possible axial orientations of a bone bolt engaged with a compression washer with a threaded nut portion, locking sleeve, and elongated plate according to one embodiment of the apparatus of the present invention; and

FIG. 13 is a schematic view illustrating one embodiment of the apparatus of the present invention in use as part of a spinal fixation system.

### DETAILED DESCRIPTION OF THE INVENTION

The present invention will now be described with occasional reference to the specific embodiments of the invention. Unless otherwise defined, all terms used herein have the same meaning as commonly understood by one of ordinary skill in the art to that this invention belongs. The terminology used in the description of the invention herein is for describing particular embodiments only, and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing units of measure as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

As used throughout this disclosure, the term “substantially” means significantly, considerably, or for the most part, and when used with reference to the shape terms “conical,” “spherical,” “elliptical,” “square,” “rectangular,” and the like, is intended to contemplate shapes that may deviate slightly, or comprise minor variations or interruptions. For example, when the term “substantially spherical” is used, it is intended to encompass a shape according to a common definition of spherical that refers to a feature having the form of a sphere or of one of its segments (e.g., hemisphere, arc), wherein a sphere or a portion of a sphere is defined by a three-dimensional surface wherein every point on the surface is equidistant from the center, including shapes that are generally dome-like but not perfectly spherical such that most but not every point on the surface is equidistant from the center.

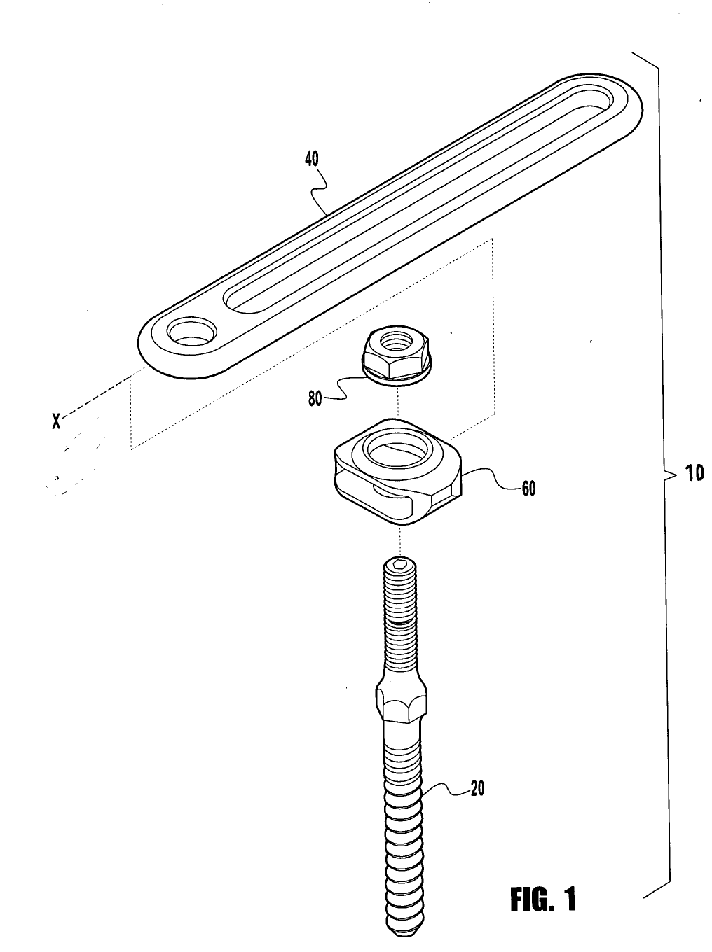

Turning now to description of the bone fixation apparatuses of the invention, FIG. 1 illustrates an embodiment of the apparatus of the present invention designated generally by the reference numeral “10.” Referring to FIG. 1, there can be seen bone bolt 20, an elongated plate 40, a locking sleeve 60, and a compression washer 80, aligned for engagement according to the invention. Each of these components, including their various embodiments, as more fully described herein, comprise materials that are satisfactory for use in achieving bone fixation, and more particularly for implantation into patients in need of such devices. Of course it will be understood that the materials useful for providing the bone fixation system and its components according to the invention are not limited, and can vary according to the materials known or developed within the art.

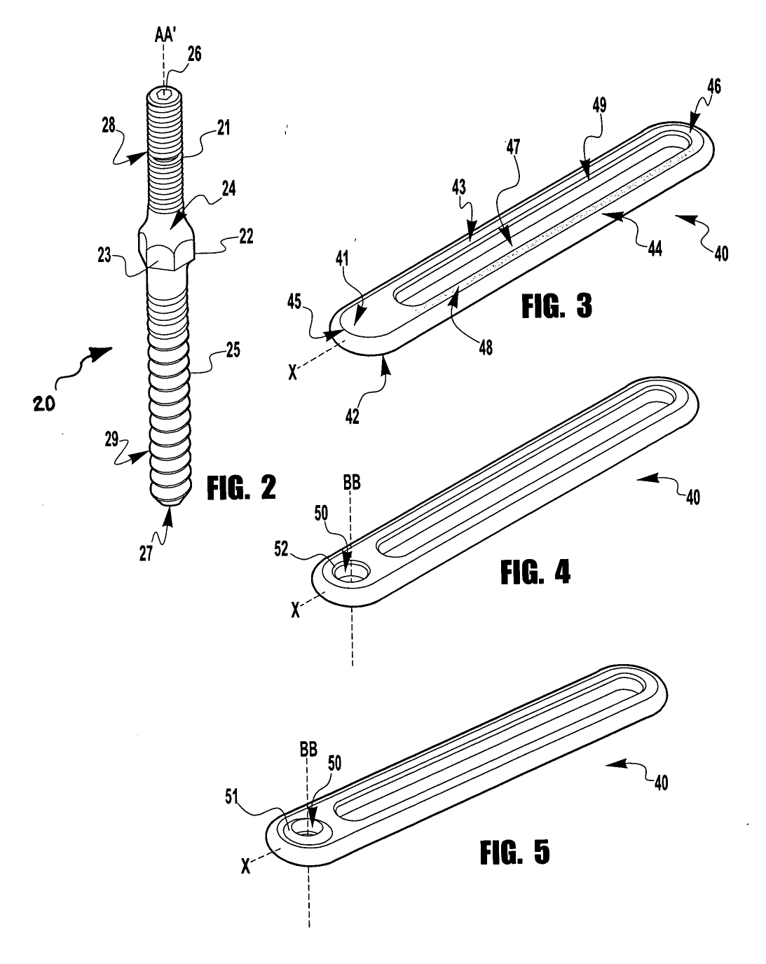

Referring to FIG. 2, a first component of the bone fixation apparatus 10 is a bone bolt 20 having an axis AA′ and comprising a first end portion 21, a central portion 22, and a second end portion 25, wherein the diameter of the central portion 22 is greater than the diameters of the first end portion 21 and the second end portion 25. In some embodiments, as illustrated in FIG. 2, the first end portion 21 has a diameter that is less than the diameter of the second end portion 25. Of course, in alternate embodiments, the first end portion 21 has a diameter that is equal to or greater than the diameter of the second end portion 25. According to the illustrated embodiment, the bone bolt 20 also comprises a hexagonal tool socket 26 for receiving an allen wrench or like tool to aid in manipulation of the bone bolt 20 for surgical installation into bone. The illustrated bone bolt 20 also comprises an optional void 27 visible at the terminus of the second end portion 25 which passes partially or completely through the length of at least the second end portion 25. According to alternate embodiments of the invention, the bone bolt 20 may lack one or both of the features including the hexagonal tool socket 29, and the void 27. Further, the bone bolt 20 may comprise additional features that are known in the art to be useful or satisfactory for facilitating the manipulation of the bone bolt 20 or its surgical installation into bone.

Referring again to FIG. 2, the first end portion 21 of the illustrated embodiment of the bone bolt 20 comprises along at least a portion of its length a fine thread 28. According to various embodiments of the bone bolt 20 of the present invention, a fine thread 28 on the first end portion 21 is useful for engagement with a nut (not shown) to facilitate locked engagement between the bone bolt 20 and other components of the fixation apparatus 10. The second end portion 25 of the illustrated embodiment of the bone bolt 20 comprises along at least a portion of its length a coarse thread 29. According to various embodiments of the bone bolt 20 of the present invention, a coarse thread 29 on the second end portion 21 is useful for insertion into and engagement with a bone (not shown), such as the pedicle or other portion of a vertebra. A variety of fine and course threads are well known in the art, more particularly with respect to the bone bolt art, and even more particularly with respect to bone bolts for spinal fixation. Applicant's disclosure is not intended to limit the possible thread configurations that may be employed with respect to embodiments of the bone bolt 20 of the present invention.

Again referring to FIG. 2, the middle portion 22 of the bone bolt 20 has a portion that comprises flat faces 23, wherein according to the illustrated embodiment the flat faces 23 are arranged around the periphery of the central portion 22 to form a hexagonal shape. The central portion 22 of the illustrated embodiment of the bone bolt 20 also comprises an upper surface 24 which is substantially spherical convex in shape. As will be further described herein, this spherical convex upper surface 24 of the central portion 22 of the bone bolt 20 is adapted for engagement with other portions of the bone fixation apparatus 10 to enable the desired orientation and locking of the apparatus within bone tissue of a patient in need of the same.

Alternate embodiments of the bone bolt 20 comprise a central portion 22 that lacks one or both of the features that include the spherical convex upper surface 24 and the flat faces 23. For example, in alternate embodiments, the central portion 22 may lack flat faces 23 around its periphery, or the number of flat faces 23 may vary, and may number from 1, to 2, to 3, to 4, to 5, to 7, to 8 or more, arranged around the periphery of the central portion 22. Likewise, in alternate embodiments, the upper surface 24 of the central portion 22 of the bone bolt 20 may be substantially flat, or may be convex elliptical.

Turning now to a second component of the bone fixation apparatus 10, FIG. 3, FIG. 4 and FIG. 5 show various illustrated embodiments of an elongated plate 40. FIG. 3 shows an elongated plate 40 having a longitudinal axis X, and comprising an upper surface 41, a lower surface 42, first and second outer edges 43 and 44, first and second ends 45 and 46, and an opening 47 bounded by said first and second outer edges 43 and 44, and said first and second ends 45 and 46. According to the illustrated embodiment, the opening 47 has a length that is less than the length of the elongated plate 40, and is generally centered within the outer boundaries of the elongated plate 40. According to the illustrated embodiments of FIGS. 3, 4 and 5, the opening 47 is slot-like in shape, extends longitudinally through the center-line of the elongated plate 40, and comprises semicircular ends. In alternate embodiments, the opening 47 may have a length that extends along a greater or lesser length of the longitudinal axis X of the elongated plate 40, and the opening 47 may be positioned parallel to the longitudinal axis X but not centered between the first and second outer edges 43 and 44. The illustrated embodiment of the elongated plate 40 also comprises a bevel or concavity on one or both of interior edges 49 of the opening 47. In various embodiments, this concavity on the interior edge 49 of the opening 47 may be conical, spherical, or of some other shape. According to some embodiments of the elongated plate 40, neither of the interior edges 49 comprises a bevel or concavity.

In the illustrated embodiment, the elongated plate 40 is a single piece in which the corners where the first and second outer edges 43 and 44, and the first and second ends 46 and 47 converge, are curved. Likewise, in the illustrated embodiment, the first and second outer edges 43 and 44, and the first and second ends 45 and 46 are rounded to form a convex domed shape around the periphery of the elongated plate 40. As will be appreciated, alternate embodiments according to the invention may lack one or both of the curved corners and rounded peripheral edges, and may embody alternate forms. For example, according to some alternate embodiments, the corners of the elongated plate 40 may be squared. In yet other embodiments, the peripheral edges of the elongated plate 40 are substantially flat, or are concave and comprise a groove along all or a portion of one or more of the first and second outer edges 43 and 44, and the first and second ends 45 and 46 of the elongated plate 40. In yet other alternate embodiments, the elongated plate 40 may comprise more than a single piece; for example, the plate may comprise two or more rod-like members (not shown) that can be engaged with one another at one or both ends of each rod to form a plate that has first and second outer edges 43 and 44, first and second ends 45 and 46 that may be opened or closed, wherein the outer edges and ends bound an opening between the two rod-like members that is parallel to the rods. According to such embodiments, the inner and outer edges of the rod-like members comprise one or more individual or combinations of features described herein in connection with the elongated plate 40 embodiments illustrated in FIG. 3, FIG. 4 and FIG. 5.

In some embodiments, the elongated plate 40, as illustrated in FIG. 3, comprises on at least a portion of at least one of its upper and lower surfaces 41 and 42 an optional locking enhancement means 48, which is shown as a granular or rough surface texture. It will be appreciated that the locking enhancement means 48 is intended to provide additional friction between one or more of the upper and lower surfaces 41 and 42 of the elongated plate 40, and objects that come into contact with such upper and lower surfaces 41 and 42, such as the locking sleeve 60, described herein, as well as bone and other structures in a patient into whom the bone fixation apparatus 10 of the invention is introduced. The locking enhancement means 48 may take alternative forms, such as cuts or grooves arranged in patterns such as in parallel or cross hatches, bumps, ridges, or any of a number of other textural features, on all or a portion of one or both of the upper and lower surfaces 41 and 42 of the elongated plate 40. The locking enhancement means 48 may be integral with the surface of the elongated plate 40, or may comprise material that is applied to the surface of the elongated plate 40 by spray or other application means. In some embodiments, the elongated plate 40 lacks locking enhancement means 48.

Referring to FIG. 4 and FIG. 5, the illustrated embodiments of the elongated plate 40 comprise a through-hole 50 having an axis BB at one of the first and second ends 45, 46 of the elongated plate 40. The embodiment illustrated in FIG. 4 comprises within the inner perimeter of the through-hole 50 on the upper surface 41 of the elongated plate 40 a bevel or concavity 52, which may be conical, spherical or have some other shape. According to some embodiments of the elongated plate 40, as described more fully herein, the concave inner perimeter 52 on at least one of the upper and lower surfaces 41 and 42 of the through-hole 50 of the elongated plate 40 has a substantially spherical shape that is particularly adapted for engagement with the convex upper surface 24 of the central portion 22 of an embodiment of the bone bolt 20 according to the invention. The concave inner perimeter 52 of the through-hole 50, and more particularly a spherical concave inner perimeter 52, is adapted for permitting engagement of the bone bolt 20 within the through-hole 50 at one of a wide range of positions, wherein the axis of the bone bolt 20 may be aligned in parallel with the axis BB of the through-hole, or may be oriented so as to transect the axis BB of the through-hole, depending on the angle at which the bone bolt 20 is inserted into the through-hole 50 and engages at its spherical convex upper surface 24 with the concave inner perimeter 52 of the through-hole 50.

The embodiment illustrated in FIG. 5 comprises at the outer perimeter of the through-hole 50 on the upper surface 41 of the elongated plate 40 a substantially spherical convex outer perimeter 51. According to other embodiments of the elongated plate 40 illustrated in FIG. 5, the elongated plate 40 further comprises a concave inner perimeter 52 on the lower surface 42 of the through-hole 50. In one such embodiment, the concave inner perimeter 52 has a substantially spherical shape that is particularly adapted for engagement with the convex upper surface 24 of the central portion 22 of an embodiment of the bone bolt 20 according to the invention.

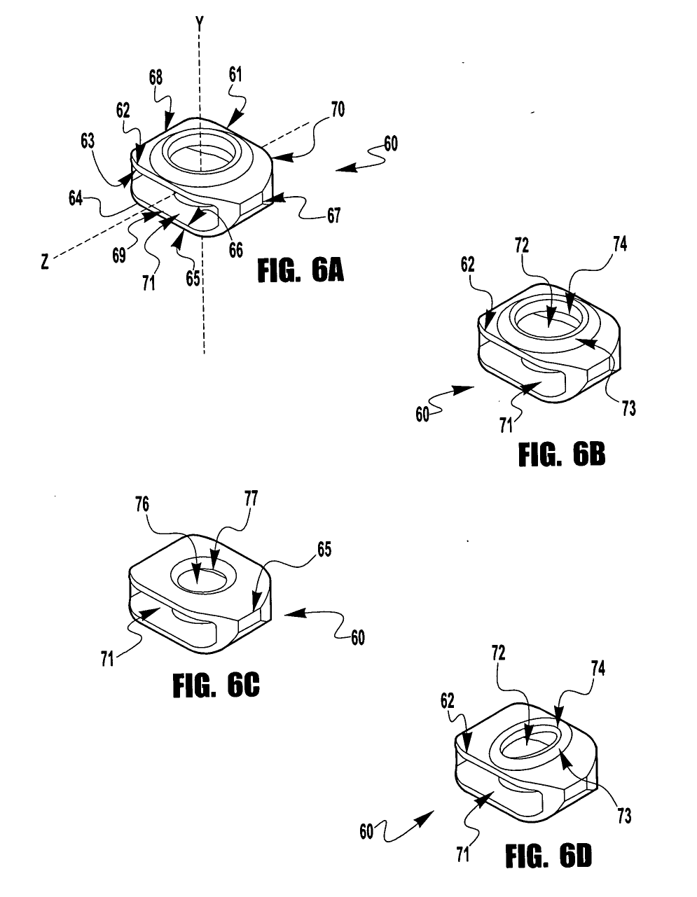

Turning now to a third component of the bone fixation apparatus 10, FIGS. 6A through D show various embodiments of a locking sleeve 60 according to the invention. Referring to FIG. 6A, the locking sleeve 60 comprises an upper wall 61 and a lower wall 64, each of which comprise an exterior surface 62 and 65, and an interior surface 63 and 66. The locking sleeve 60 also comprises first and second sidewalls 67 and 68, and first and second opened ends 69 and 70 that provide an aperture 71 having a central axis Z through the locking sleeve 60. As depicted in FIGS. 6A through D, the locking sleeve 60 has a generally rectangular shape, with a first width defined as the distance between the first and second sidewalls 67 and 68, and a second width defined as the distance between the first and second opened ends 69 and 70. According to the embodiment illustrated in FIGS. 6A through D, the first width is greater than the second width. It will be appreciated that in alternate embodiments, the locking sleeve 60 may have a substantially square shape, such that the first and second widths are essentially equal. In yet other embodiments, the dimensions of the first and second widths may further vary to provide a rectangular device wherein the second width is greater than the first width. As can be seen in FIG. 6A, the upper and lower walls 61, 62 and the sidewalls 67, 68 have an essentially uniform thickness, and each of the sidewalls 67 and 68 intersects with the adjacent portions of the upper and lower walls 61 and 62 to form rounded corner edges. In alternate embodiments, the corner edges may be more or less rounded, or may form a right angle. Likewise, the thickness of each of the walls of the locking sleeve 60 may vary from one another.

Referring to FIG. 6B, the illustrated locking sleeve 60 comprises a through-hole 72 in its upper wall 62 comprising a substantially convex outer perimeter 73 and a conical concave interior perimeter 74, wherein the diameter of the through-hole 72 on the exterior surface 62 of the upper wall 61 is greater than the diameter on the interior surface 63 of the upper wall 61. In alternate embodiments, the interior perimeter 74 of the upper wall through-hole 72 is cylindrical, having substantially straight walls, such that the diameter of the through-hole 72 on the exterior surface 62 of the upper wall 61 is approximately equal to the diameter on the interior surface of the upper wall 61. Generally, the upper wall through-hole 72 has a maximum diameter on the exterior face 62, and a minimum diameter on the interior face 63, wherein the diameter of the interior face 63 does not exceed the diameter on the exterior face 62.

Referring to FIG. 6C, the illustrated locking sleeve 60 comprises a through-hole 76 in its lower wall 65 having a substantially spherical concave interior perimeter 77, wherein the diameter of the through-hole 76 on the exterior surface 65 of the lower wall 64 is greater than the diameter on the interior surface 66 of the lower wall 64. Generally, according to the embodiments of the locking sleeve 60 as illustrated in FIGS. 6a-D, the lower wall through-hole 76 has a maximum diameter on the exterior surface 65, and a minimum diameter on the interior face 66, wherein the diameter of the lower wall through-hole 76 on the interior surface 66 is always less than the diameter on the exterior surface 65. As previously described in connection with an embodiment of an elongated plate 40 comprising a through-hole 50, the spherical concave inner perimeter 77 of the through-hole 76 is adapted for permitting engagement of the bone bolt 20 within the through-hole 76 at one of a wide range of positions, wherein the axis AA′ of the bone bolt 20 may be aligned in parallel with the axis Y of the through-hole 76, or may be oriented so as to transect the axis Y of the through-hole 76, depending on the angle at which the bone bolt 20 is inserted into the through-hole 76 and engages at its spherical convex upper surface 24 with the concave inner perimeter 77 of the through-hole 76.

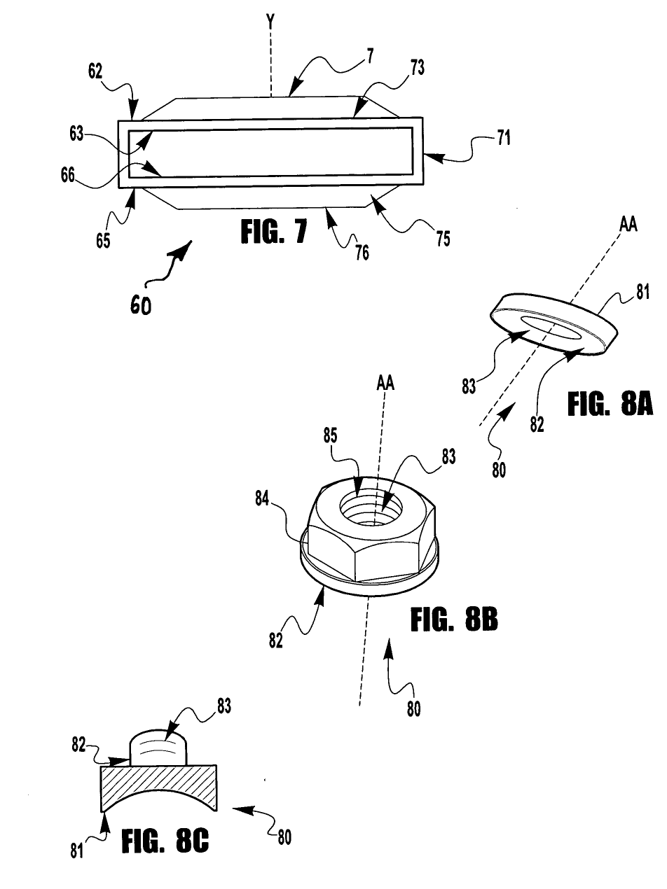

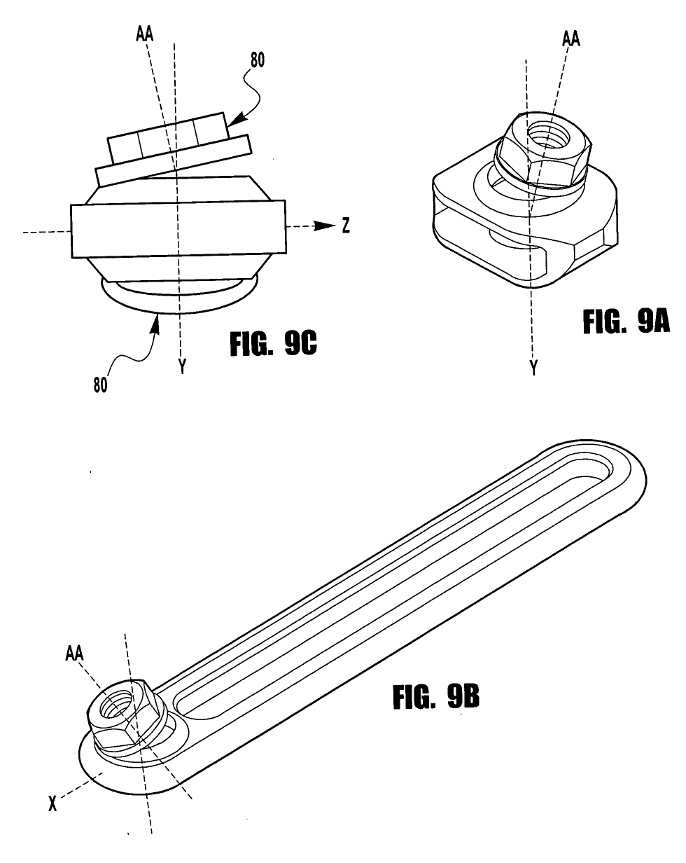

FIG. 7 shows an illustration of one alternate embodiment of a locking sleeve 60, wherein the lower wall through-hole 76 comprises a substantially convex outer perimeter 75 and a conical concave interior perimeter 78, wherein the exterior diameter of the through-hole 76 on the exterior surface 65 of the lower wall 64 is greater than the diameter on the interior surface 66 of the lower wall 64. In alternate embodiments, the interior perimeter 77 of the lower wall through-hole 76 is cylindrical, having substantially straight walls, such that the diameter of the through-hole 76 on the exterior surface 65 of the lower wall 64 is approximately equal to the diameter on the interior surface 66 of the lower wall 64. Generally, according to the embodiments as illustrated in FIG. 7, the lower wall through-hole 76 has a maximum diameter on the exterior surface 65, and a minimum diameter on the interior surface 66, wherein the diameter of the interior surface 66 does not exceed the diameter on the exterior surface 65.

The upper and lower wall through-holes 72 and 76 are generally spherical according to various embodiments of the invention, and share a common central axis Y. In various embodiments of the locking sleeve 60, one or both of the minimum and maximum diameters the upper and lower wall through-holes 72 and 76 are approximately equal. In yet another embodiment, the upper wall through-hole 72 is elliptical in shape, as illustrated in FIG. 6D, wherein the long axis of the elliptical through-hole 72 is aligned with the axis Z of the aperture 71. According to the embodiments shown in FIGS. 6A-D, the through holes are generally centered between the first and second sidewalls 67 and 68, and the first and second opened ends 69 and 70. In alternate embodiments, the through-holes are not centered on the upper and lower surfaces 62 and 65 of the locking sleeve 60.

Turning now to a fourth component of the bone fixation apparatus 10, FIGS. 8A-C show compression washers 80 according to the invention. FIG. 8A shows a compression washer having a substantially disc-like shape comprising a flat upper surface 81 and comprising on its lower surface 82 a substantially spherical concavity, and having a central hole 83 with a central axis AA for receiving therethrough a first end portion 21 of a bone bolt 20 according to one or more of the embodiments described herein. The lower surface concavity 82 of the compression washer 80 is configured to contact and engage with one or more of the substantially spherical convex outer perimeters on one of the embodiments of the locking sleeve 60 and elongated plate 40. In some embodiments, the compression washer 80 has a central hole 83 having a diameter sufficient to receive therethrough the first end portion 21 of a bone bolt 20. In yet other embodiments, the compression washer 80 has a central hole 83 having a different diameter, for example, a diameter sufficient to receive therethrough the second end portion 25 of a bone bolt 20. FIG. 8B shows one alternate embodiment of a compression washer 80 comprising a nut portion 84 that comprises a threaded interior 85. A cross sectional view of the compression washer of FIG. 8B is shown in FIG. 8C. The nut portion 84 is configured to receive the first end portion 21 of a bone bolt 20 and reversibly engage with the same by cooperation between the threaded interior 85 of the nut portion 84 of the compression washer 80 and threads on the first end portion 21 of the bone bolt 20.

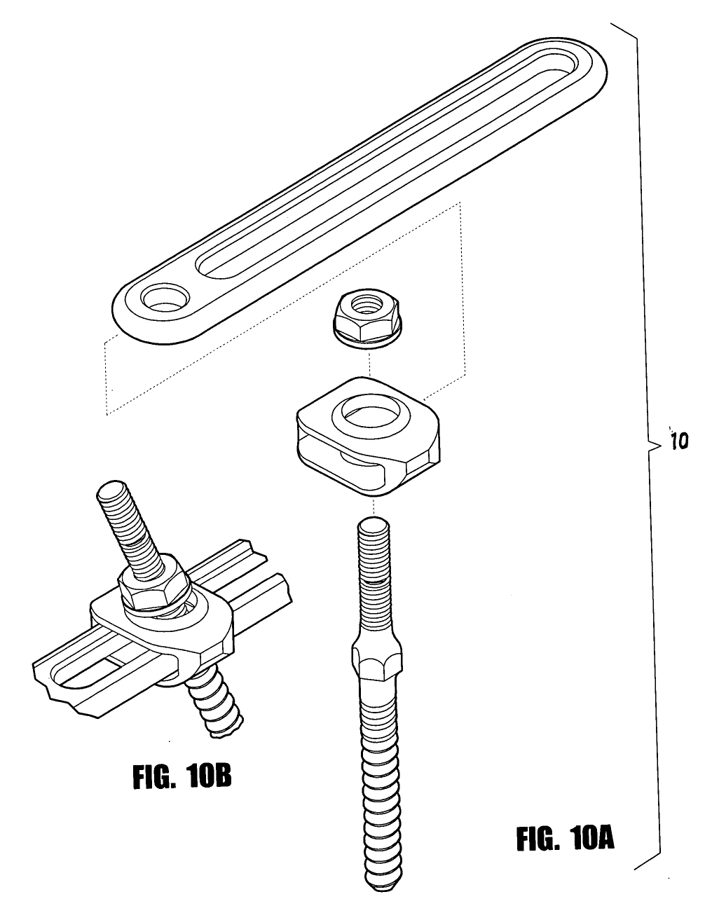

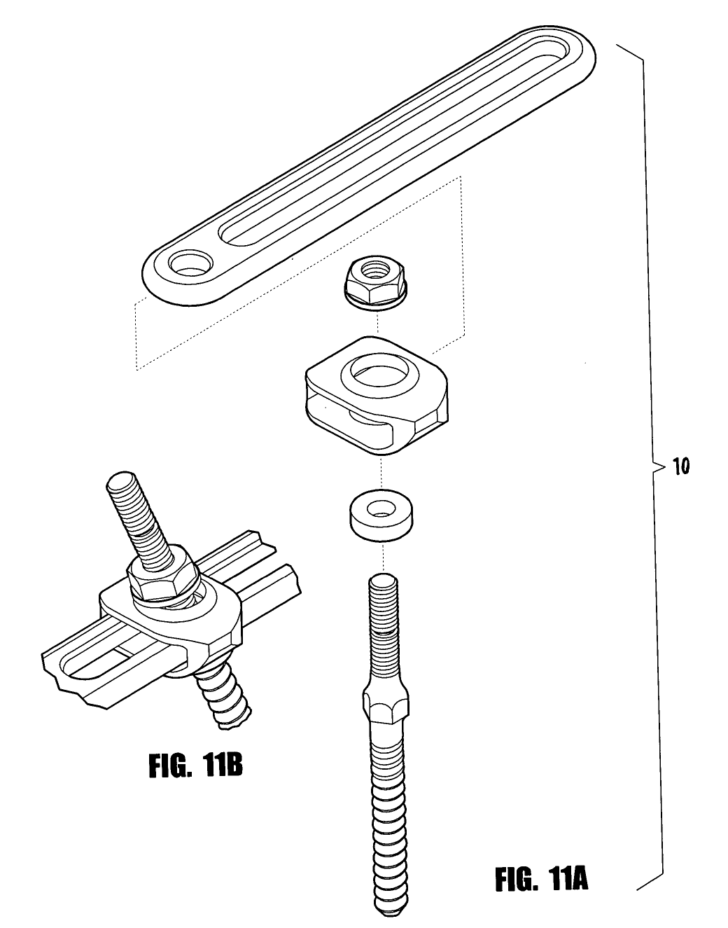

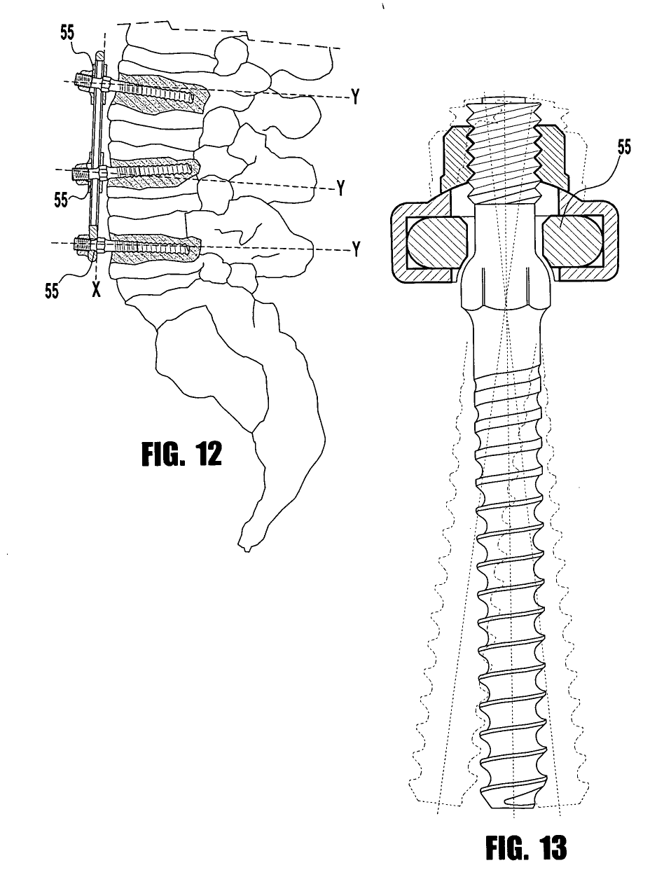

Turning now to description of the use of the components of the bone fixation apparatus 10, FIGS. 10A and 11A illustrate various components of the system engaged for use according to the instant invention. Referring to FIG. 10A, one of the first and second ends 45 and 46 of the elongated plate 40 is passed through the aperture 71 in a locking sleeve 60 such that the central axis Y of the locking sleeve 60 through-holes 72 and 76 is aligned with the center of the opening 47 in the elongated plate 40. Once the elongated plate 40 and the locking sleeve 60 are engaged, the first end portion 21 of a bone bolt 20 comprising a convex spherical upper surface 24 on its central portion 22 can be passed through the aligned through-holes 72 and 76 and opening 47.

Prior to locking of the fixation apparatus 10, the position of the bone bolt 20 and locking sleeve 60 can be slidably adjusted along the length of the elongated plate 40 to occupy any position between the ends of the opening 47. Likewise, the position of the bone bolt 20 can be adjusted such that the bone bolt 20 axis AA′ can be aligned with the locking sleeve through-hole axis Y, or the bone bolt 20 can be rotated at its engagement locus between the convex spherical upper surface 24 on its central portion 22 and the spherical concavity within the lower wall 64 through-hole 76 to assume a position wherein the bone bolt 20 axis AA′ transects the locking sleeve through-hole axis Y. According to the various embodiments of the bone fixation apparatus 10, the range of degrees of angulation (i.e., the angle, in degrees, between the axes Y and AA) is from 0.2, 0.4, 0.6, 0.8, 1, 2, 3, 4, 5, 6, 7, 8, 9, 10, 11, 12, 13, 14, 15 or more. In some embodiments wherein the locking sleeve 60 comprises an elliptical upper though-hole 72 (as described and illustrated in connection with FIG. 6D), the range of degrees of angulation may be greater than with a locking sleeve 60 that comprises a circular upper though-hole 72. According to the embodiment of the fixation device shown in FIG. 10A, good results have been obtained wherein the range of angulation between the axes Y and AA is 10°.

Once the desired position of the locking sleeve 60 and bone bolt 20 combination has been selected on the elongated plate 40, and the position of the bone bolt 20 relative to the through-hole axis Y has been achieved, the device is reading for locking, as shown in FIG. 10 B. Using appropriate tools known in the art for manipulation and installation of bone bolts and related parts, a compression washer 80 having a threaded nut portion 82 is engaged with the first end portion 21 of the bone bolt 20 by cooperation between the nut threads 85 and the bone bolt 20 threads 28. The fixation apparatus 10 is locked when the concave spherical lower surface 82 of the compression washer 80 engages with the spherical convex outer perimeter 73 of the upper wall through-hole 72. The interaction between these two surfaces, in cooperation with the interaction between the convex spherical upper surface 24 on the central portion 22 of the bone bolt 20 and the spherical concavity 77 within the lower wall through-hole 76, allow for a high-degree of rotational freedom of the bone bolt 20 within the aligned elongated plate 40 and locking sleeve 60.

When the components are engaged, the concave spherical lower surface 82 of the compression washer 80 interfits with the spherical convex outer perimeter 73 of the upper wall through-hole 72 and delivers a compressive force to the upper surface 62 of the locking sleeve 60 to achieve locking. When the fixation apparatus 10 is locked, the upper surface 62 of the locking sleeve 60 can be observed to bow along the aperture 71 axis Z such that the locking sleeve 60 assumes a figure-8-like, or generally lemniscate shape. In some embodiments, the assembly of a bone fixation apparatus 10 as described above will involve the use of an elongated plate 40 that has a through-hole as described in connection with one of FIG. 4 or FIG. 5.

As previously described herein, in some embodiments according to the invention the interior perimeter of the upper wall though-hole 74 has a concave shape that such as would be defined by an inverted cone. This concavity aids in providing maximal rotational freedom of the bone bolt 20 relative to the upper wall through-hole 72. In alternate embodiments, the interior perimeter of the upper wall though-hole 74 is not concave, and has a straight shape such as would be defined by cylinder, with walls that are parallel to the through-hole axis Y.

In yet another alternate embodiment, a bone fixation apparatus 10 is shown in FIG. 11 A, wherein an elongated plate 40 and a locking sleeve 60 are engaged. A bone bolt 20 comprising a flat upper surface 24 on its central portion 22, is first passed through the central hole 83 in a compression washer 80, oriented with its lower surface concavity 82 facing upwards; thereafter, the bone bolt 20 can be passed through the aligned through-holes 72 and 76 and opening 47 of the elongated plate and locking sleeve combination. The apparatus is further combined as described above in connection with the embodiments illustrated in FIG. 10A and 10B wherein the desired position of the locking sleeve 60 along the elongated plate 40 is chosen, and the axial orientation of the bone bolt 20 is selected, the opposing upper compression washer 80 is engaged with the first end portion 21 of the bone bolt 20, and the components are locked. In this configuration, tightening of the nut portion 84 of the upper compression washer 80 is expected to result in the application of opposing forces on each of the upper and lower surfaces 62 and 65 of the locking sleeve 60, such that both the upper and lower surfaces 62 and 65 of the locking sleeve 60 can be observed to bow along the aperture axis Z such that the locking sleeve 60 assumes a figure-8-like, or generally lemniscate shape.

When the fixation apparatus 10 is used in a patient, as depicted in FIG. 12, the components according to the various embodiments of the invention are installed into a patient, wherein at least two bone bolts 20 are used. In use, the bone bolts 20 are first threaded into appropriate structures of selected bones, usually spinal vertebra, which are usually adjacent. A key advantage to the apparatuses of the present invention is that each bone bolt can be installed in an optimal position to best ensure stability. Once installed, an appropriate length elongated plate 40 is selected, and a number of locking sleeves equal to the number of bone bolts is selected. The appropriate number of locking sleeves are engaged in series on the elongated plate, either at the same time or one at a time, the combination of plate and one or more locking sleeves is aligned with a first installed bone bolt, a locking sleeve being placed over said bone bolt, and positioning of the sleeve and bone bolt determined. The process is repeated for each bone bolt, followed by engagement with compression washers and locking.

In alternate embodiments, a locking sleeve having a hole at one end, as described herein, is used for attachment to one of the installed bone bolts. In yet other embodiments, bone fixation apparatuses according to the embodiment described in connection with FIG. 11A and FIG. 11B may be employed, wherein a different embodiment of bone bolt 20 is used with an additional compression washer.

The embodiments described above are examples of preferred embodiments and are not intended to limit the scope of the claims set forth below. Variations to the inventions described herein, including alternate embodiments not specifically described, are quiet possible and are encompassed by the claims as understood by one of ordinary skill in the art. Indeed, the claimed inventions have their broad and ordinary meaning as set forth below in the claims.

We claim:

1. A bone fixation locking system, comprising:
a locking sleeve comprising opposing upper and lower walls each comprising interior and exterior faces, opposing first and second sidewalls, and opposing first and second opened ends defining an aperture through said sleeve, said upper wall comprising a through-hole, said through-hole defined on the exterior face of the upper wall by a spherical convex outer perimeter, and said lower wall comprising a through-hole, wherein the upper and lower wall through-holes of the locking sleeve share a common axis.

2. The bone fixation locking system according to claim 1, wherein the lower wall through-hole is defined on the exterior face of the lower wall by a spherical concave inner perimeter.

3. The bone fixation locking system according to claim 1, comprising:
a compression washer comprising a concave spherical lower surface for engagement with the spherical convex outer perimeter of the exterior face upper wall through-hole.

4. A bone fixation locking system according to claim 1, wherein the compression washer comprises a threaded nut portion.

5. A bone fixation locking system according to claim 1, wherein the upper and lower wall through-holes of the locking sleeve have approximately the same diameter.

6. A bone fixation locking system according to claim 1, wherein the upper and lower wall through-holes of the locking sleeve have different diameters.

7. A bone fixation locking system according to claim 1, wherein the upper wall through-hole on the exterior face comprises a concave inner perimeter that is either conical or spherical.

8. A bone fixation locking system according to claim 7, wherein the upper wall through-hole on the exterior face comprises a conical concave inner perimeter.

9. A bone fixation locking system according to claim 1, wherein the locking sleeve, in use, can be compressed along an axis that is parallel to said first and second opposing sidewalls and is substantially perpendicular to the axis of the through-holes to acquire a lemniscate shape.

10. A bone fixation locking system according to claim 9, wherein the upper wall is displaced along the through-hole axis toward the lower wall.

11. A bone fixation locking system, comprising:
a locking sleeve comprising opposing upper and lower walls each comprising interior and exterior faces, opposing first and second sidewalls, and opposing first and second opened ends defining an aperture through said sleeve, said upper wall comprising a through-hole, said through-hole defined on the exterior face of the upper wall by a spherical convex outer perimeter, and said lower wall comprising a through-hole, said through-hole defined on the exterior face of the lower wall by a spherical convex outer perimeter, wherein the upper and lower wall through-holes of the locking sleeve share a common axis; and
first and second compression washers, each comprising a concave spherical lower surface for engagement with the spherical convex outer perimeters of the exterior face upper and lower wall through-holes.

12. A bone fixation locking system according to claim 11, wherein at least one of said compression washers comprises a threaded nut portion.

13. A bone fixation locking system according to claim 11, wherein the upper and lower wall through-holes of the locking sleeve have approximately the same diameter.

14. A bone fixation locking system according to claim 11, wherein the upper and lower wall through-holes of the locking sleeve have different diameters.

15. A bone fixation locking system according to claim 11, wherein the upper and lower wall through-holes on the exterior faces each comprise a concave inner perimeter that is either conical or spherical.

16. A bone fixation locking system according to claim 15, wherein the upper and lower wall through-holes on the exterior faces each comprise a conical concave inner perimeter.

17. A bone fixation locking system according to claim 11, wherein the locking sleeve, in use, can be compressed along an axis that is substantially perpendicular to the axis of the through-holes to acquire a lemniscate shape.

18. A bone fixation locking system according to claim 17, wherein the upper and lower walls are each displaced along the through-hole axis toward a center position located between the upper and lower walls.

19. A bone fixation apparatus, comprising:
an elongated plate having a longitudinal axis, and comprising upper and lower surfaces, first and second outer edges parallel to said longitudinal axis, first and second ends defining a longitudinal dimension, and an opening bounded by said first and second outer edges and said first and second ends, and oriented along the longitudinal axis;
a locking sleeve for slidably engaging with the elongated plate, the locking sleeve comprising opposing upper and lower walls each comprising interior and exterior faces, opposing first and second sidewalls, and opposing first and second opened ends defining an aperture through said sleeve, said upper wall comprising a through-hole, said through-hole defined on the exterior face of the upper wall by a spherical convex outer perimeter, and said lower wall comprising a through-hole, said through-hole defined on the exterior face of the lower wall by a spherical concave inner perimeter, wherein the upper and lower wall through-holes of the locking sleeve share a common axis, and wherein said through-hole axis intersects at substantially right angles the longitudinal axis of the elongated plate when said locking sleeve is engaged with said elongated plate; and
a compression washer comprising a concave spherical lower surface for engagement with the spherical convex outer perimeter of the exterior face upper wall through-hole.

20. The bone fixation apparatus according to claim 19, wherein the compression washer comprises a threaded nut portion.

21. The bone fixation apparatus according to claim 19, wherein the upper and lower wall through holes of the locking sleeve have approximately the same diameter.

22. The bone fixation apparatus according to claim 19, wherein the upper and lower wall through holes of the locking sleeve have different diameters.

23. The bone fixation apparatus according to claim 19, wherein the upper wall through-hole on the exterior face comprises a concave inner perimeter that is either conical or spherical.

24. The bone fixation apparatus according to claim 19, wherein the upper wall through-hole on the exterior face comprises a conical concave inner perimeter.

25. The bone fixation apparatus according to claim 19, wherein the locking sleeve, in use, can be compressed along an axis that is substantially perpendicular to the axis of the through-holes to acquire a lemniscate shape.

26. The bone fixation apparatus according to claim 25, wherein the upper wall is displaced along the through-hole axis toward the lower wall.

27. The bone fixation apparatus according to claim 19, wherein the elongated plate comprises a locking enhancement means.

28. The bone fixation apparatus according to claim 27, wherein the locking enhancement means comprises texture on all or a portion of at least one of the upper and lower surfaces of the elongated plate.

29. The bone fixation apparatus according to claim 19, wherein the opening comprises a concave interior perimeter along at least one of the upper and lower surfaces of the elongated plate, and wherein such concavity is either conical or spherical.

30. The bone fixation apparatus according to claim 29, wherein the opening comprises a concave interior perimeter along the upper and lower surfaces of the elongated plate.

31. The bone fixation apparatus according to claim 19, wherein the opening comprises a width that is approximately equal to the diameter of at least one of the locking sleeve through-holes.

32. The bone fixation apparatus according to claim 19, wherein the elongated plate comprises a through-hole at one of its ends.

33. The bone fixation apparatus according to claim 32, wherein the through-hole in the elongated plate comprises a spherical convex exterior perimeter on the upper surface of the elongated plate, and a spherical concave interior perimeter on the lower surface of the elongated plate.

34. The bone fixation apparatus according to claim 33, wherein the through-hole in the elongated plate comprises a concave conical or spherical interior perimeter on the upper surface of the elongated plate.

35. The bone fixation apparatus according to claim 34, wherein the through-hole in the elongated plate comprises a concave conical interior perimeter on the upper surface of the elongated plate.

36. The bone fixation apparatus according to claim 19, wherein the opening comprises a length that is sufficient to accommodate at least two spaced-apart bone bolts.

37. A bone fixation apparatus, comprising:
at least one bone bolt comprising a threaded first end portion, a central portion comprising a spherical convex upper surface adjacent to the first end portion, and a threaded second end portion;
an elongated plate having a longitudinal axis and comprising upper and lower surfaces, first and second outer edges parallel to said longitudinal axis, first and second ends defining a longitudinal dimension, and an opening for receiving the threaded first end portion of the at least one bone bolt, said opening bounded by said first and second outer edges and said first and second ends, and oriented along the longitudinal axis;
a locking sleeve for slidably engaging with the elongated plate, the locking sleeve comprising opposing upper and lower walls each comprising interior and exterior faces, opposing first and second sidewalls, and opposing first and second opened ends defining an aperture through said sleeve, said upper wall comprising a through-hole, said through-hole defined on the exterior face of the upper wall by a spherical convex outer perimeter, and said lower wall comprising a through-hole, said through-hole defined on the exterior face of the lower wall by a spherical concave inner perimeter, wherein the upper and lower wall through-holes of the locking sleeve share a common axis, and wherein said through-hole axis intersects at substantially right angles the longitudinal axis of the elongated plate when said locking sleeve is engaged with said elongated plate; and
a compression washer comprising a concave spherical lower surface for engagement with the spherical convex outer perimeter of the exterior face upper wall through-hole.

38. The bone fixation apparatus according to claim 37, wherein the threaded first end portion of the at least one bone bolt is adapted for receiving a nut, and wherein the threaded second end portion is adapted for surgical implantation into bone tissue;

39. The bone fixation apparatus according to claim 37, wherein said compression washer comprises a threaded nut portion for engagement with the threaded first end portion of at least one of the at least one bone bolt.

40. The bone fixation apparatus according to claim 37, wherein the diameters of the threaded first end and central portions of at least one of the at least one bone bolt are different, such that only the first end portion is insertable through the opening in the elongated plate and the through-holes in the locking sleeve, and the central portion of the bone bolt is unable to pass through the opening in the elongated plate and the through-holes in the locking sleeve.

41. The bone fixation apparatus according to claim 39, wherein at least one of the at least one bone bolt is adapted for polyaxial positioning when inserted through the opening in the elongated plate and the through-holes in the locking sleeve.

42. The bone fixation apparatus according to claim 37, comprising at least two bone bolts, wherein at least one bone bolt is adapted for polyaxial positioning when inserted through the opening in the elongated plate and the through-holes in the locking sleeve, and wherein at least one bone bolt is not adapted for polyaxial positioning when inserted through the opening in the elongated plate and the through-holes in the locking sleeve.

43. The bone fixation apparatus according to claim 37, wherein the central portion of at least one of the at least one bone bolt comprises a hexagonal shape.

44. The bone fixation apparatus according to claim 37, wherein the upper and lower wall through-holes of the locking sleeve have approximately the same diameter.

45. The bone fixation apparatus according to claim 37, wherein the upper and lower wall through-holes of the locking sleeve have different diameters.

46. The bone fixation apparatus according to claim 37, wherein the upper wall through-hole on the exterior face comprises a concave inner perimeter that is either conical or spherical.

47. The bone fixation apparatus according to claim 46, wherein the upper wall through-hole on the exterior face comprises a conical concave inner perimeter.

48. The bone fixation apparatus according to claim 37, wherein the locking sleeve, in use, can be compressed along an axis that is substantially perpendicular to the axis of the through-holes and substantially parallel to the longitudinal axis of the elongated plate, to acquire a lemniscate shape.

49. The bone fixation apparatus according to claim 48, wherein the upper wall is displaced along the through-hole axis toward the lower wall.

50. The bone fixation apparatus according to claim 37, wherein the elongated plate comprises a locking enhancement means.

51. The bone fixation apparatus according to claim 50, wherein the locking enhancement means comprises texture on all or a portion of at least one of the upper and lower surfaces of the elongated plate.

52. The bone fixation apparatus according to claim 37, wherein the opening in the elongated plate comprises a length that is sufficient to accommodate at least two spaced-apart bone bolts and comprises a conical or spherical concave interior perimeter along at least one of the upper and lower surfaces of the elongated plate, and wherein the elongated plate comprises a through-hole at one of its ends, wherein the through-hole comprises a conical or spherical concave interior perimeter on at least one of the upper and lower surfaces of the elongated plate.

53. A bone fixation apparatus, comprising:
an elongated plate having a longitudinal axis and comprising upper and lower surfaces and having a longitudinal dimension, and a central opening parallel to the longitudinal axis and having a length that is less than the longitudinal dimension, said opening adapted to receive at least two bone bolts;
a locking sleeve comprising opposing upper and lower walls each comprising an exterior and an interior face, opposing first and second sidewalls, and an aperture through which the elongated plate can be inserted, said upper and lower walls each comprising through-holes that share a common axis and are adapted for receiving a portion of a bone bolt, wherein said through-hole axis intersects at substantially right angles with the longitudinal axis of the elongated plate when said elongated plate is inserted through said aperture, and wherein the through-holes are adapted with means to permit positioning of an inserted bone bolt along a range of axes that transect the through-hole axis, and wherein at least the upper wall through-hole comprises on the exterior face a domed convex outer perimeter;
at least one bone bolt comprising a threaded first end portion adapted for insertion through the opening in the longitudinal plate and the locking sleeve through holes, a central portion that cannot be passed through the opening in the longitudinal plate and the locking sleeve through-holes and comprising a spherical convex upper surface adjacent to the first end portion, and a threaded second end portion adapted for insertion into bone; and
at least one compression washer comprising a concave spherical lower surface for engagement with the spherical convex outer perimeter of the exterior face upper wall through-hole.
